(19) United States
(12) Patent Application Publication
(10) Pub. No.: US 2026/0256216 A1
(43) Pub. Date: Sep. 3, 2026

(54) Aerosol-Generating Device with Anti-Residue Surface

(71) Applicant: JT International SA, Geneva (CH)

(72) Inventors: Patrick Debergh, Cressier (CH); Christel Henrich, Neuchâtel (CH)

(73) Assignee: JT International SA, Geneva (CH)

(21) Appl. No.: 18/880,993

(22) PCT Filed: Jul. 6, 2023
(86) PCT No.: PCT/EP2023/068655

§ 371 Date: Jan. 3, 2025

(30) Foreign Application Priority Data
Jul. 8, 2022 (EP) 22183719.8

(51) Int. Cl. A24F 40/85; A24F 40/20; A24F 40/46; A61L 2/088; A61L 2/10; A61L 103/25

(52) U.S. Cl. CPC A24F 40/85; A24F 40/46; A61L 2/088; A61L 2/10; A24F 40/20; A61L 2103/25; A61L 2202/11; A61L 2202/17

(57) ABSTRACT

An aerosol-generating device includes a heating cavity adapted to receive at least part of an aerosol-generating article, and an electrical heating system including at least one heater configured to heat an aerosol-generating article received in the heating cavity. The heating cavity includes at least one active surface having photocatalytic properties, as an anti-residue surface.

### TECHNICAL FIELD OF THE INVENTION

The present invention relates to aerosol-generating devices.

### BACKGROUND OF THE INVENTION

Aerosol-generating devices, also commonly known as heat-not-burn (HNB) electronic cigarettes, are nowadays increasingly used as a substitution for regular cigarettes.

An aerosol-generating device typically comprises a heating cavity adapted to receive at least part of a consumable aerosol-generating article inserted therein, and a heater for heating said heating cavity and hence the article contained therein.

The aerosol-generating article contains a tobacco substrate comprising an aerosol-forming substance (such as glycerine and/or propylene glycol) which vaporises during heating and creates a vapour that extracts nicotine and flavour components from the tobacco substrate. The aerosol-forming substance is heated to between 200 and 400° C., which is below the normal burning temperatures of a conventional cigarette.

Volatile compounds and aerosol released upon heating of the tobacco substrate become deposited on surfaces of the aerosol-generating device. Also, pieces or particles of the aerosol-generating articles themselves, such as pieces or particles from their wrapper or of the substrate, may come off when the article is manipulated or in use.

All such residues are particularly deposited on the lateral and/or bottom surfaces of the heating cavity. They may be further accumulated and/or partially removed by the friction of inserted aerosol-generating articles.

Such residues hinder optimal use of the aerosol-generating device. When accumulated on the walls of the heating cavity, they can diminish or block the required air flow of the device. The residues may also affect the optimal flavour sensation of the aerosol. Indeed, the contamination pieces or particles may impart an unpleasant or bitter flavour to a user. Also, the heater may be damaged depending on how and where the residues are deposited.

Nowadays, users generally clean their devices themselves, using dedicated cleaning tools such as brushes and according to a cleaning frequency suggested by the manufacturer based on statistical knowledge of the mean contamination levels of heating cavities in function of the frequency of use of the device. However, predictions regarding contamination may not be reliable enough, as they may be distorted by individual using habits, and the heater may be damaged by either too much or too few cleaning operations with such cleaning tools. This solution also puts a heavy burden on the user.

It is also known to partially reduce contamination by using a pyrolysis method, in which a heater is heated to a temperature sufficiently high to burn any residues. However, this method has not proven to be sufficiently effective.

There is thus a need to improve management of residues inside aerosol-generating devices, to improve the user's experience while vaping and in maintenance operations and to more globally enhance sustainability of said device.

### SUMMARY OF THE INVENTION

This is achieved with an aerosol-generating device as defined in claim 1, comprising:
a heating cavity extending along a longitudinal axis and provided with an opening at one insertion end, the heating cavity being adapted to receive at least part of an aerosol-generating article inserted through said opening, and
an electrical heating system comprising an electrical power source and at least one heater powered by said electrical power source and configured to heat an aerosol-generating article received in said heating cavity, the aerosol-generating device being characterized in that the heating cavity comprises at least one active surface having photocatalytic properties.

Residues formed in the heating cavity of an aerosol-generating device are generally carbon-based residues. These residues are for example volatile compounds and aerosol released upon heating of the tobacco substrate contained in aerosol-generating articles and/or particles of the aerosol-generating articles themselves. The invention is based on the fact that such carbon-based residues, whether in solid or gaseous form, may become decomposed and degraded under photocatalytic effect.

When activated by electromagnetic radiation, a photocatalytic material creates electron-hole pairs, which generated free radicals, thus allowing an oxidation-reduction reaction through which organic molecules become absorbed and decomposed by the photocatalytic material.

The heating cavity of an aerosol-generating device according to the invention comprises at least one active surface with such photocatalytic properties (i.e. made of a photocatalytic material), as an anti-residue surface.

Residues accumulating on the active surface(s) of the cavity are degraded by photocatalytic reaction, upon illumination. Once degraded, they are prevented from adhering to said active surface(s) or, if already deposited on said surface(s), are easily removable.

An active surface of the heating cavity may delimit said heating cavity (i.e. for instance it may form a lateral and/or a bottom surface of the heating cavity) and/or it may be housed by said heating cavity (i.e. for instance it may be part of an element protruding inside the cavity).

The at least one active surface is typically part of a photocatalytic structure formed of a photocatalytic material, with the photocatalytic structure delimiting the heating cavity and/or housed in said cavity.

In the present context, a photocatalytic structure might either be a rigid or self-supporting element or part of such element, for example a layer thereof. As an alternative, a photocatalytic structure might also be a coating or a film.

The photocatalytic material may for example comprise at least one photocatalyst among any one of: TiO₂, ZnO, SnO₂, Fe₂O, WO₂, In₂O₃, C₃N₄.

According to an example, at least 20%, preferably at least 50%, more preferably at least 75% by weight of the photocatalytic material may be formed of a photocatalyst.

According to an example, said at least one active surface forms at least part of a lateral surface and/or a bottom surface of the longitudinal heating cavity, preferably at least 20% of the lateral and bottom surfaces of the longitudinal heating cavity, still more preferably at least 50% of the lateral and bottom surfaces of the longitudinal heating cavity.

In particular, the active surface may cover determined longitudinal sections of the cavity.

In the present application, a lateral surface of the cavity is understood as a surface delimiting the cavity in a direction perpendicular to the longitudinal axis, and a bottom surface is understood as a surface delimiting the cavity in the longitudinal direction, at its end opposite to the insertion end.

A lateral surface of the heating cavity is for example a generally cylindrical surface, for example of circular section. A bottom surface is for example a flat surface orthogonal to the longitudinal axis.

Active surfaces are advantageously provided in locations of the cavity which are more prone to accumulation of residues, typically in the vicinity of the heater.

The heater may for example comprise a heater body and, if relevant, at least one heating element such as a heating electrode or an induction coil.

According to an example, the heating cavity may be at least partially surrounded or delimited by the heater. For example, the heater body may take the form of a sleeve, with the heating cavity being formed in the inner volume of said sleeve.

As an alternative, the heater may protrude inside the heating cavity. For example, the heater body may be in the form of a stick protruding from the bottom surface of the cavity, inside said cavity.

The at least one active surface may be at least partially aligned with the heater, and particularly with the heater body, in a transversal direction orthogonal to the longitudinal axis. In other words, at least part of the active surface is advantageously located in a longitudinal section of the cavity surrounded by the heater.

According to an example, the heater body may be at least partially formed of a photocatalytic material and may at least partially delimit the cavity. In such case, the active surface may be a surface of the heater body itself.

Or the heater body may be coated with a photocatalytic coating: the active surface is then a surface of said coating.

Electromagnetic radiations, required for triggering the photocatalytic reaction, may be provided by a light source located outside the aerosol-generating device, for example by the sun. In such case, the aerosol-generating device may be so configured that light rays issuing from said external light source impinge on the active surface. According to an example, the device may comprise an outer body having a window configured to transmit light from the light source situated outside said device to said active surface.

According to another example, the aerosol-generating device may comprise at least one light source configured to illuminate at least a portion of said active surface.

In the present application, a light source should be understood as a source of electromagnetic radiations.

Said light source may for example emit electromagnetic radiations having wavelengths in the UV and/or visible part (preferably blue part) of the electromagnetic spectrum.

Said light source may for example be chosen among one of: a LED, a semiconductor laser, a superluminescent diode (SLED).

Said light source may also be a pulsed light source, such as a pulsed LED or a pulsed semiconductor laser.

According to an example, several light sources may be arranged so as to illuminate different zones of the heating cavity.

At least one light source may be provided at one or each longitudinal end of the cavity. In addition or as an alternative, at least one light source may be located at the center of the heating cavity or several light sources may be distributed along the heating cavity.

According to a preferred embodiment, a single light source may be provided, said light source being configured to illuminate the whole cavity surface or at least a critical part thereof comprising the active surface.

Advantageously, said single light source may be located at or near the insertion end of the cavity.

If possible, said light source may also advantageously be shifted with respect to the heater in the longitudinal direction of the cavity.

The invention further concerns a cleaning device for cleaning a heating cavity of an aerosol-generating device as previously defined, said cleaning device comprising a rod and at least one light source fixed to said rod.

Advantageously said at least one light source is configured to emit light having a wavelength of less than 485 nm. A light source emitting wavelengths of less than 485 nm, preferably less than 450 nm, even more preferably less than 380 nm, have a high cleaning efficiency when combined with photocatalytic materials. The highest efficiency is obtained with light sources emitting in the UV region, i.e. having wavelengths below 380 nm.

Such cleaning device allows activation of the photocatalytic effect for controlled degradation of residues formed in the heating cavity. The cleaning device is particularly helpful in combination with aerosol-generating devices having no integrated light source or dedicated means such as a window for allowing external light to illuminate the at least one photocatalytic structure. However, the cleaning device may also be used to enhance the photocatalytic reaction in combination with other light source(s) internal or external to the aerosol-generating device.

According to an advantageous example, the cleaning device may further comprise at least one cleaning brush fixed to said rod. The cleaning brush allows removing the residues detached from the surface of the heating cavity and decomposed by photocatalytic effect.

The invention further concerns a method for reducing the contamination of a heating cavity of an aerosol-generating device as previously defined, comprising at least the step of illuminating said photocatalytic structure before, during or after the use of said aerosol-generating device, with a light source or by light provided by the environment of said device.

According to an example, the method may further comprise the steps of:
introducing the cleaning device as defined previously into said longitudinal heating cavity;
operating the at least one light source of said cleaning device in front of said at least one active surface.

It is to be understood that the different examples mentioned hereabove can be realized singly or in any technically compatible combinations. In particular, the aforementioned technical features and those to be explained in the following can be used not only in the combinations indicated, but also in other combinations or alone, without departing from the scope of the present invention.

### BRIEF DESCRIPTION OF THE DRAWINGS

FIG. 1 is an overall view of an aerosol-generating device according to a first embodiment of the present invention;

FIG. 2 illustrates an aerosol-generating device according to a second embodiment of the present invention, in which a plurality of light sources are distributed along the longitudinal direction of the heating cavity;

FIG. 3 illustrates an aerosol-generating device according to a third embodiment of the present invention, in which an active surface extends on a limited longitudinal section of the cavity;

FIG. 4 illustrates an aerosol-generating device according to a fourth embodiment of the present invention, in which the heater body forms a photocatalytic structure;

FIG. 5 illustrates an aerosol-generating device according to a fifth embodiment of the present invention, in which the heater has the form of a stick protruding inside said cavity;

FIG. 6 illustrates an aerosol-generating device according to a sixth embodiment of the present invention, having an outer body provided with a window;

FIG. 7 illustrates the cooperation of an aerosol-generating device according to a seventh embodiment of the present invention with a cleaning device configured to trigger the photocatalytic reaction on the active surface of the cavity.

### DETAILED DESCRIPTION OF THE INVENTION

The present invention will be described with respect to particular embodiments and with reference to the appended drawings, but the invention is not limited thereto. In the drawings, which are only schematic, the size of some of the elements may be exaggerated and not drawn on scale for illustrative purposes. The dimensions and the relative dimensions do not correspond to actual reductions to practice of the invention.

In the different figures, elements that are functionally identical are designated by similar references with increments of 100.

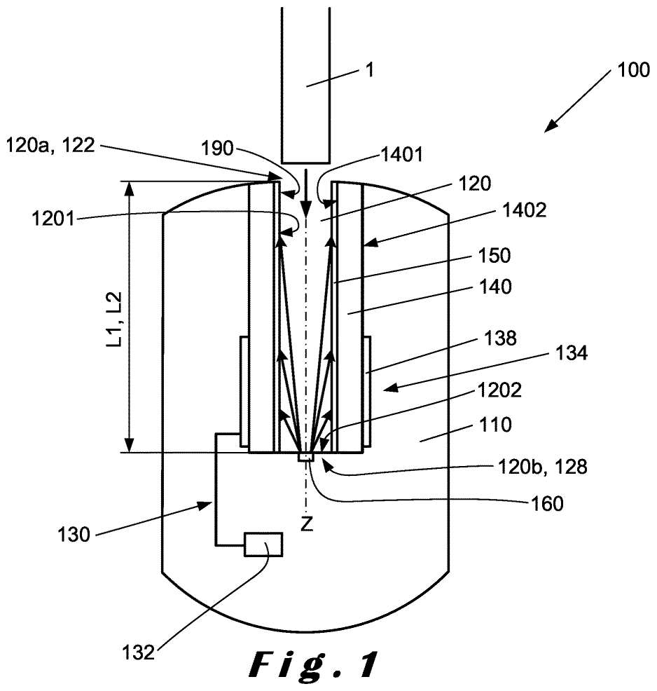

FIG. 1 illustrates an aerosol-generating device 100 according to a first embodiment of the present invention. The device 100 comprises an outer body 110 of any adapted shape, housing a longitudinal heating cavity 120 (hereafter also named cavity) extending along an axis Z, of length L1, and provided with an opening 122 at one insertion end 120a. The heating cavity 120 is adapted to receive at least part of an aerosol-generating article 1 inserted through its opening 122.

The outer body 110 further houses an electrical heating system 130 comprising an electrical power source 132 and at least one heater 134 powered by said electrical power source 132, with the heater 134 being configured to heat the aerosol-generating article 1 received in the heating cavity 120.

The heater 134 comprises at least a heater body 140, here in the form of a longitudinal metal sleeve of axis Z and length L2 substantially equal to L1. In the illustrated example, the heater 134 further comprises at least one heating element 138, such as an electrode or an induction coil, possibly integrated in a layer, contacting the heater body. In the figure, the heating element 138 is contacting the outer surface 1402 of said heater body 140. More generally, said heating element(s) may be arranged at an inner and/or outer side of the heater body 140.

On the inner surface 1401 of the heater body 140, advantageously on said entire inner surface 1401, is provided a photocatalytic structure 150, here in the form of a coating made of photocatalytic material.

According to alternative embodiments, the photocatalyst structure 150 may also be a photocatalytic film deposited on the inner surface 1401 of the heater body 140, or a rigid element arranged inside the heater body 140.

Furthermore, although the photocatalytic structure 150 is defined with respect to FIG. 1 as covering the entire periphery of the cavity 120, such structure 150 may also form just a part of a lateral surface 1201 and/or a bottom surface 1202 of the cavity. Still further, different photocatalytic structures may also be provided inside the heating cavity 140.

The photocatalytic material constituting the photocatalytic structure 150 advantageously comprises at least one photocatalyst among any one of: TiO₂, ZnO, SnO₂, Fe₂O, WO₂, In₂O₃, C₃N₄.

Preferably, at least 20%, more preferably at least 50%, still more preferably at least 75% by weight of said photocatalytic material is formed of a photocatalyst of one or more of the above-mentioned types.

According to this first embodiment, the heating cavity 120 is defined in the inner volume of the heater body 140 and delimited transversally by said photocatalytic coating 150, the surface 190 of said coating 150 therefore forming the lateral surface 1201 of the heating cavity 120.

At its end 120b opposite to the insertion end 120a, the cavity 120 is closed by a bottom wall 128 the surface of which forms the bottom surface 1202 of the cavity, typically a flat surface extending transversally (in the entire present description, the adjective transversal or the adverb transversally shall designate elements or directions that are orthogonal or extend orthogonally to the longitudinal direction Z).

Due to the material from which it is formed, surface 190 forms an active surface with photocatalytic properties.

When an aerosol-generating article 1 is inserted in the heating cavity 120 and the device 100 is actuated by the user, an electrical current is supplied to the heating element 138 by the electrical power source 132. The heating element 138 is heated, and the heat is transmitted by thermal conduction to the heater body 140 and consequently to the article 1 surrounded by said heater body 140.

The aerosol-generating article 1 typically comprises a tobacco substrate containing an aerosol-forming substance (such as glycerine and/or propylene glycol), in a generally cellulose-based wrapper. However, any other consumable article may be used, which comprises a material capable of generating an inhalable aerosol when heated.

Upon heating, the aerosol-forming substance contained in the tobacco substrate of the article 1 vaporises and creates a vapour that extracts nicotine and flavour components from said tobacco substrate.

Released volatile compounds and aerosol become deposited on the surfaces 1201, 1202 of the heating cavity 120, forming residues. Also, pieces or particles of the aerosol-generating articles 1 themselves, such as pieces or particles from their wrapper or of the substrate, may come off when the article 1 is manipulated or in use and form further residues on the surfaces 1201, 1202 of the cavity 120.

Residues being generally carbon-based composites, they react with the photocatalyst(s) present on the active surface 190 upon illumination.

Electromagnetic radiations, required for triggering the photocatalytic reaction, may be provided by at least one light source 160 provided in the aerosol-generating device 100 and emitting light inside the cavity 120 to illuminate at least a portion of said active surface 190.

In the embodiment of FIG. 1, one single light source 160 is provided at the bottom end 120b of the cavity 120. As illustrated, the light source 160 may for example be integrated in bottom wall 128 of the cavity 120.

As an alternative, several light sources 160 may be arranged so as to illuminate different zones of the heating cavity 120.

Each light source 160 may for example be chosen among one of: a LED, a semiconductor laser, a superluminescent diode (SLED).

Each light source 160 may for example emit electromagnetic radiations having wavelengths in the UV and/or in the visible part, preferably the blue part, of the electromagnetic spectrum.

A light source 160 may also be a pulsed light source, such as a pulsed LED or a pulsed semiconductor laser.

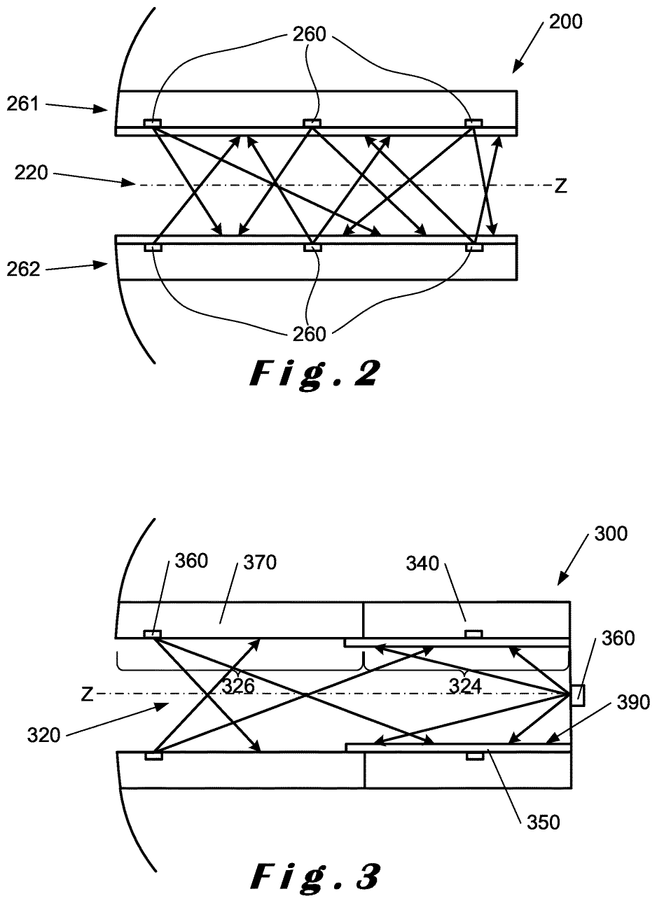

FIG. 2 illustrates an aerosol-generating device 200 according to a second embodiment of the present invention, in which a plurality of light sources are distributed along the longitudinal direction Z of the heating cavity 220.

In the illustrated embodiment, in particular, multiple rows 261, 262 of light sources 260 are provided along the longitudinal direction Z of the cavity 220, with the light sources 260 of each row being preferably distributed at regular intervals.

As illustrated, the light sources 260 of different rows 261, 262, preferably of all rows, may be aligned transversally.

Furthermore, the different rows 261, 262 may be arranged in an axial symmetry around axis Z.

As an alternative, light sources 260 of adjacent rows 261, 262 may also be staggered.

FIG. 3 illustrates an aerosol-generating device 300 according to a third embodiment of the present invention, in which an active surface 390 extends on a limited longitudinal section of the cavity 320 instead of on the entire length thereof.

In this third embodiment, the heater does not extend along the entire length L1 of the heating cavity 320.

As illustrated, the heating cavity 320 is surrounded by the heater body 340 on a first longitudinal section 324, here at its bottom side, and by a non-heating body 370 on a second longitudinal section 326, here at its insertion side.

Residues being particularly important in the vicinity of the heater body 340, a photocatalytic structure 350 is provided primarily facing said heater body 340 and/or proximate thereto.

In the embodiment of FIG. 3, the photocatalytic structure is in the form of a coating 350 applied on the first longitudinal section 324 and eventually on a limited portion of the second section 326 adjacent to the first section.

The active surface 390 of said coating 350 forms part of the lateral surface of the cavity 320.

As illustrated, the device may further comprise light sources 360, arranged in an appropriate manner along the cavity, for illuminating said active surface 390.

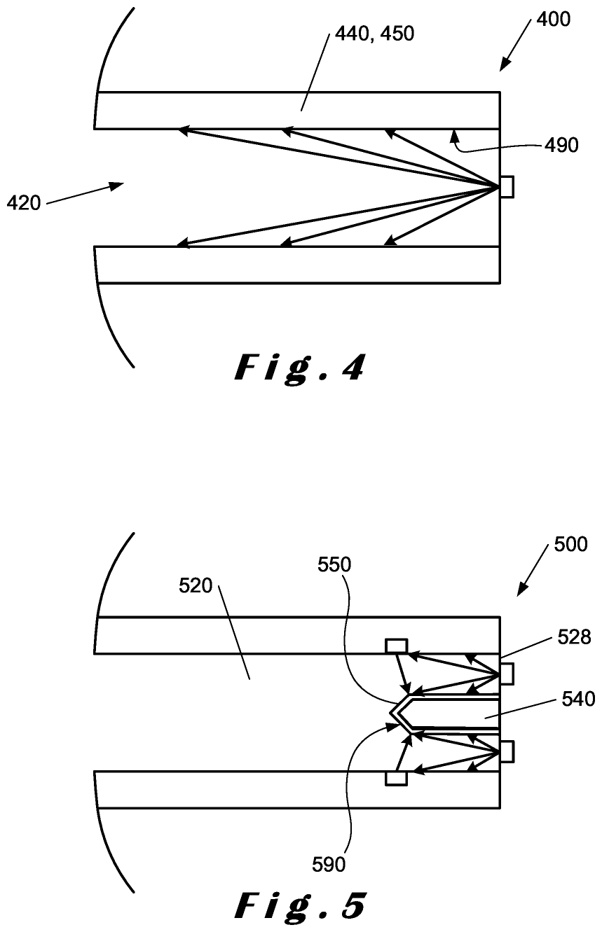

FIG. 4 illustrates an aerosol-generating device 400 according to a fourth embodiment of the present invention, in which the heater body 440 forms a lateral wall delimiting the heating cavity 420 transversally, and in which said heater body 440 itself forms the photocatalytic structure 450.

The heater body 440 is here formed of a photocatalytic material. In particular, photocatalyst are integrated in the material of the heater body 440 (or at least the material of an inner layer of the heater body 440), so that the inner surface thereof forms an active surface 490 having photocatalytic properties.

The active surface 490 here forms the lateral surface of the longitudinal cavity 420.

FIG. 5 illustrates an aerosol-generating device 500 according to a fourth embodiment of the present invention, in which the heater body 540 has the form of a stick protruding from the bottom wall 528 of the heating cavity 520, inside said cavity 520.

In a manner similar to what has been described previously with reference to FIG. 1, 2 or 3, a photocatalytic coating 550 is applied on the heater body 540, thus forming an active surface 590 having photocatalytic properties on top of said heater body.

As an alternative embodiment, the photocatalytic coating 550 may be replaced by any photocatalytic structure having similar properties.

As another alternative embodiment (not shown), in a manner similar to what has been described previously with reference to FIG. 4, a photocatalyst may also be contained in the material of the heater body 540 itself, in which case the active surface is the surface of said heater body.

As an alternative or in addition, the lateral surface of the longitudinal cavity 520 or part thereof may also be provided as an active surface.

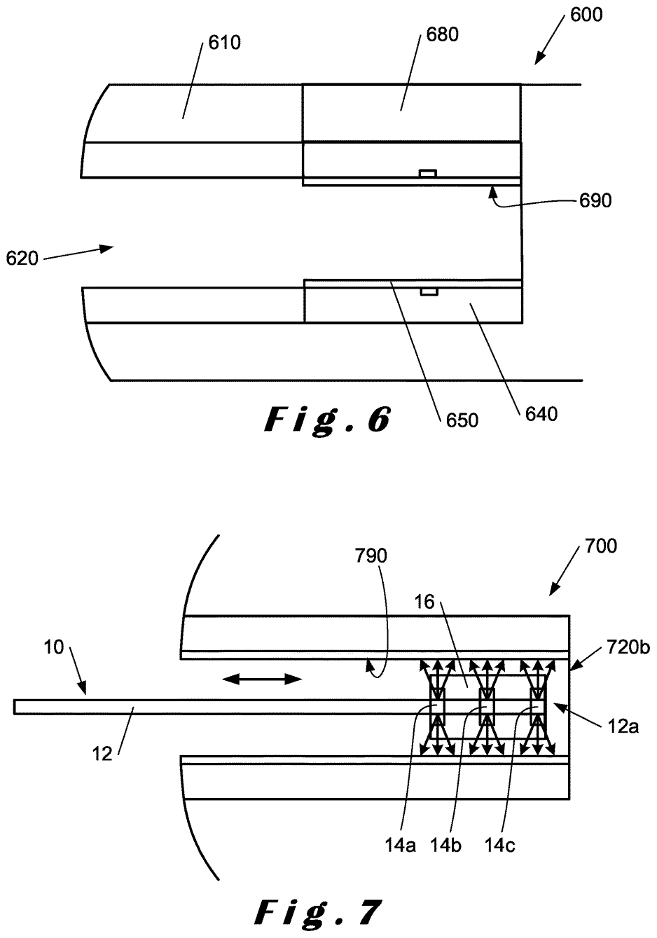

FIG. 6 illustrates an aerosol-generating device 600 according to a sixth embodiment of the present invention, in which the outer body 610 is provided with a window 680 configured to transmit sun light or any light from a light source situated outside said device to the active surface(s) 690 formed in the heating cavity 620. Although the heating cavity 620 has been illustrated in a configuration similar to that described previously with reference to FIG. 3, this shall not be seen as limiting, the only requirement being that the elements located between the window 680 and the heating cavity 620, if any, be able to transmit light towards the cavity. In the illustrated example, for example, the heater body 640 and photocatalytic structure 650 shall be made of transparent material.

Depending on the configuration and needs, the device 600 may or may not have one or more additional integrated light source(s).

FIG. 7 illustrates an aerosol-generating device 700 according to a seventh embodiment of the present invention, provided with an active surface 790 in the cavity 720 but having no light source and no dedicated means to transmit external light towards said active surface.

Activation of the photocatalytic reaction between the residues and the photocatalyst present on the active surface 790 formed inside the cavity 720 is implemented with a cleaning device 10 as illustrated.

The cleaning device 10 is designed and dimensioned to be introduced into the heating cavity 720, preferably to reach the bottom end 720b thereof.

The cleaning device 10 comprises a rod 12 and at least one light source, preferably a plurality of light sources 14a, 14b, 14c as illustrated, fixed to said rod 12, advantageously at a distal end 12a thereof. In a preferred embodiment at least one light source 14a, 14b, 14c is configured to emit light in the blue range of the electromagnetic spectrum, i.e. having wavelengths between 450 nm and 485 nm. More preferably the light source emits in the violet region, i.e. having wavelengths between 380 nm and 450 nm. It is even more preferable that the light source emits in the UV region, i.e. having a wavelength of less than 380 nm.

By operating the device 10 inside the cavity 720, the photocatalyst present on the active surface 790 is activated and the residues get absorbed and/or decomposed.

According to an advantageous embodiment, the cleaning device may further be provided with one or several brushes 16 at its distal end 12a, to remove the residues detached from the surfaces of the heating cavity 720 by photocatalytic reaction.

1. An aerosol-generating device comprising:
a heating cavity extending along a longitudinal axis and provided with an opening at one insertion end, the heating cavity being adapted to receive at least part of an aerosol-generating article inserted through said opening, and
an electrical heating system comprising an electrical power source and at least one heater powered by said electrical power source, the at least one heater being configured to heat an aerosol-generating article received in said heating cavity,
wherein the heating cavity comprises at least one active surface having photocatalytic properties.

2. The aerosol-generating device according to claim 1, wherein said at least one active surface is part of a photocatalytic structure formed of a photocatalytic material.

3. The aerosol-generating device according to claim 2, wherein said photocatalytic material comprises at least one photocatalyst among any one of: TiO₂, ZnO, SnO₂, Fe₂O, WO₂, In₂O₃, C₃N₄.

4. The aerosol-generating device according to claim 2, wherein at least 20%, by weight of the photocatalytic material is formed of a photocatalyst.

5. The aerosol-generating device according to claim 2, wherein said photocatalytic structure is a photocatalytic coating or film.

6. The aerosol-generating device according to claim 1, wherein said at least one active surface forms at least part of a lateral surface and/or a bottom surface of the heating cavity.

7. The aerosol-generating device according to claim 1, wherein said at least one active surface is at least partially aligned with the at least one heater in a transversal direction orthogonal to the longitudinal axis.

8. The aerosol-generating device according to claim 1, further comprising at least one light source configured to illuminate at least a portion of said at least one active surface.

9. The aerosol-generating device according to claim 8, wherein said at least one light source is an LED, a semiconductor laser, or a superluminescent diode.

10. The aerosol-generating device according to claim 8, wherein said at least one light source is a pulsed light source.

11. The aerosol-generating device according to claim 1, comprising an outer body having a window configured to transmit light from a light source situated outside said device to said at least one active surface.

12. A cleaning device comprising a rod and at least one light source fixed to said rod, said at least one light source being configured to emit light having a wavelength of less than 485 nm.

13. The cleaning device according to claim 12, further comprising at least one brush fixed to said rod.

14. A method for reducing contamination of the heating cavity of the aerosol-generating device according to claim 1, comprising at least the step of illuminating said at least one active surface before, during or after use of said aerosol-generating device with a light source or by light provided by the environment of said device.

15. The method according to claim 14, further comprising the steps of:
introducing a cleaning device into said heating cavity, the cleaning device comprising a rod and at least one light source fixed to said rod, said at least one light source being configured to emit light having a wavelength of less than 485 nm; and
operating the at least one light source of said cleaning device in front of said at least one active surface.

16. The aerosol-generating device according to claim 2, wherein at least 50% by weight of the photocatalytic material is formed of a photocatalyst.

17. The aerosol-generating device according to claim 2, wherein at least 75% by weight of the photocatalytic material is formed of a photocatalyst.

18. A system comprising:
the aerosol-generating device according to claim 1, and
a cleaning device adapted for cleaning the heating cavity of the aerosol-generating device, the cleaning device comprising a rod and at least one light source fixed to said rod, said at least one light source being configured to emit light having a wavelength of less than 485 nm.

19. The system according to claim 18, wherein said at least one light source of the cleaning device is configured to emit light having a wavelength of less than 450 nm.

20. The system according to claim 18, wherein said at least one light source of the cleaning device is configured to emit light having a wavelength of less than 380 nm.
